(19) United States
(12) Patent Application Publication
(10) Pub. No.: US 2006/0111331 A1
(43) Pub. Date: May 25, 2006

(54) Gave10 agonists for treating inflammation

(75) Inventors: Haifeng Eishingdrelo, Montville, NJ (US); Kin T. Yu, Chalfont, PA (US); Jidong Cai, Whippany, NJ (US); Paul Weissensee, Somerset, NJ (US); Jian Shen, Bridgewater, NJ (US)

Correspondence Address: ROSS J. OEHLER;AVENTIS PHARMACEUTICALS INC., 1041 ROUTE 202-206, MAIL CODE: D303A, BRIDGEWATER, NJ, 08807, US

(73) Assignee: Aventis Pharmaceuticals Inc., Bridgewater, NJ (US)

(21) Appl. No.: 10/995,997

(22) Filed: Nov. 23, 2004

(51) Int. Cl. A61K 31/58

(52) U.S. Cl. 514/176

(57) ABSTRACT

Disclosed is a method for inhibiting TNFα, IL-6, and activating GAVE10, treating, for example, inflammation thereby, the method comprising the step of administering to a subject an effective amount of a compound of the following formula: 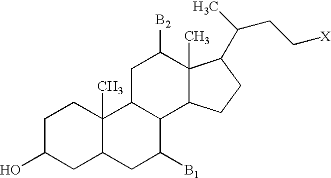 or a pharmaceutically acceptable salt, solvate, hydrate, or prodrug thereof, wherein
B₁ and B₂ are each independently —OH, (C₁-C₆)alkyl, or —H; X is Z-K;
Z is —CO or —CH₂;
K is —N(R₁)ₙ, —NR₁N(R₂)ₙ, —NR₁R₅R₂R₄, —N(R₁)ₚR₂NR₃R₄, or —N(R₁)ₚR₅R₂R₄ R₁, R₂, and R₃ are each independently (C₁-C₆)alkyl or absent; n is from 0 to 3; p is from 0 to 2;
R₄ is absent or is 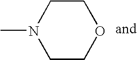
R₅ is absent or is

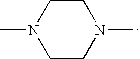

### BACKGROUND OF THE INVENTION

Human GAVE10 is a novel G-protein coupled receptor highly expressed in macrophages. Macrophages play an important role in the pathogenesis of inflammatory processes and are a major source of tumor necrosis factor α (TNFα) and other pro-inflammatory cytokines, such as the interleukins IL-1, IL-6, and IL-8. Suppressing these cytokines could be effective in reducing the activity and progression of rheumatoid arthritis, asthma, and other major inflammatory diseases which seriously impair the quality of life for many millions of people. There is currently no satisfactory therapy for many of these diseases, nor has anyone identified a compound that modulates inflammatory cytokines via GAVE10.

### SUMMARY OF THE INVENTION

The method of the invention comprises administering to a patient an effective amount of a GAVE10 agonist having the formula 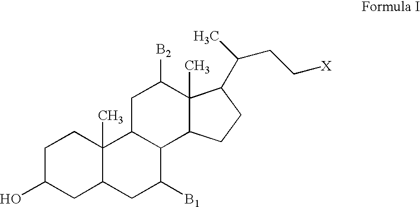
or a pharmaceutically acceptable salt, solvate, or hydrate of Formula I, wherein

B₁ and B₂ are each independently —OH, (C₁-C₆)alkyl, or —H;

X is Z-K;

Z is —CO or —CH₂;

K is —N(R₁)ₙ, —NR₁N(R₂)ₙ, —NR₁, R₅R₂R₄, —N(R₁)ₚR₂NR₃R₄, or —N(R₁)ₚR₅R₂R₄ R₁, R₂, and R₃ are each independently (C₁-C₆)alkyl or absent;

n is from 0 to 3;

p is from 0 to 2;

R₄ is absent or is

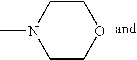

R₅ is absent or is

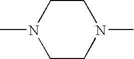

The inventors have discovered that these compounds activate GAVE10, increasing cellular cAMP and inhibiting TNFα and IL-6, the suppression of which is known to reduce inflammation. The method of the invention may be used to treat any condition in which activating GAVE10 and suppressing TNFα and IL-6 is desirable.

### BRIEF DESCRIPTION OF THE FIGURES

FIG. 1 shows the results of a Fluorometric Imaging Plate Reader (FLIPR®) assay with three compounds of the invention, Compounds A, B, and C. Compounds A, B, and C and lithocholic acid (LCA) were incubated, respectively, with PSC cells alone and PSC cells transfected with GAVE10, and the cells were then assayed in a FLIPR® assay. The graph shows that Compounds A. B, and C bind to GAVE10 to activate calcium-signaling pathways.

FIG. 2 shows the results of a cAMP assay with Compounds A, B, and C. Compounds A, B, and C and LCA were incubated, respectively, with PSC cells alone and PSC cells transfected with GAVE 10, and the cells were then assayed in a cAMP assay. The graph shows that Compounds A, B, and C activate GAVE10 to increase intracellular cAMP.

FIG. 3 shows the results of a TNFα release assay in mouse macrophages. Compounds A and B inhibited TNFα release in mouse macrophages, as did taurolithocholic acid (TLCA).

FIG. 4 shows the results of a TNFα release assay in macrophages. Deoxycholic (DCA) acids inhibited IL-6 production in mouse macrophages; the effect was more pronounced in male macrophages than in female.

### DETAILED DESCRIPTION OF THE INVENTION

The method of the invention comprises administering to a patient an effective amount of a GAVE10 agonist having the formula 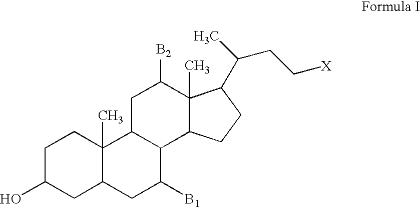
or a pharmaceutically acceptable salt, solvate, or hydrate of Formula I, wherein

B₁ and B₂ are each independently —OH, (C₁-C₆)alkyl, or —H;

X is Z-K;

Z is —CO or —CH₂;

K is —N(R₁)ₙ, —NR₁N(R₂)ₙ, —NR₁, R₅R₂R₄, —N(R₁)ₚR₂NR₃R₄, or —N(R₁)ₚR₅R₂R₄ R₁, R₂, and R₃ are each independently (C₁-C₆)alkyl or absent;

n is from 0 to 3;

p is from 0 to 2;

R₄ is absent or is

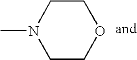

R₅ is absent or is

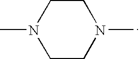

“Alkyl” in the above formula means a straight or branched, saturated or unsaturated, aliphatic radical having the number of carbon atoms indicated. “(C₁₋₆)alkyl” includes, for example, methyl, ethyl, propyl, isopropyl, butyl, sec-butyl, isobutyl, tert-butyl, vinyl, allyl, 1-propenyl, isopropenyl, 1-butenyl, 2-butenyl, 3-butenyl, 2-methylallyl, ethynyl, 1-propynyl, 2-propynyl, and the like.

The compounds of the invention have a structural backbone similar to that of bile acids, such as the primary bile acids cholic (CA) and chenodeoxycholic (CDCA) acids, and the secondary bile acids such as deoxycholic (DCA) and lithocholic (LCA) acids. Hence, they may be readily synthesized by anyone of ordinary skill in the art of chemistry. One could use the mixed anhydride method, for example, to obtain CDCA, and then subsequently reduce it with lithium aluminum hydride in anhydrous tetrahydrofuran; one could then elute with the appropriate solvents from silica gel the material thus obtained to obtain the desired compound of the invention. Variations on this method, and other methods of synthesis, as well, should be readily apparent to one of ordinary skill in the art.

Although any compound of Formula I may be used in the method of the invention, three compounds are presently preferred. In Compound A, B₁ is —H, B₂ is —OH, Z is —CO and K is 2-(Amino-ethyl)-trimethyl-ammonium, —NCH₂CH₂N⁺(CH₃)₃:

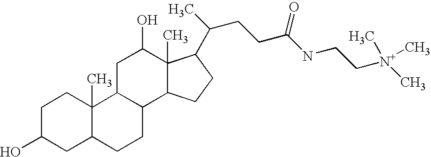

In Compound B, B₁ is —H, B₂ is —OH, Z is —CO and K is 1,4-Dimethyl-piperazine,

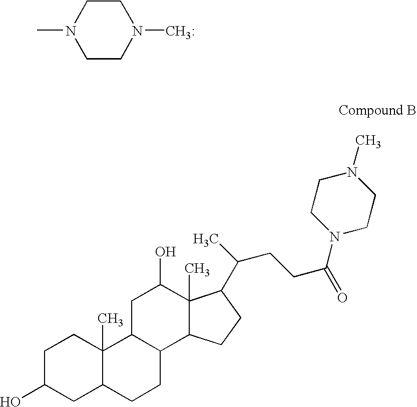

In Compound C, B₁ is —H, B₂ is —OH, Z is —CO and K is 2-Morpholin-4-yl-ethylamine,

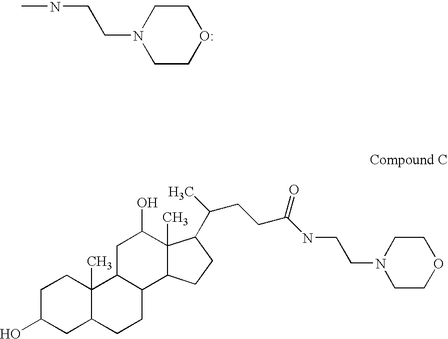

Compounds of Formulas I bind and activate GAVE10, as demonstrated by FLIPR® and cAMP assays, and as shown in FIGS. 1 and 2. The result of such binding is the inhibition of TNFα production by macrophages (FIG. 3). Compounds structurally similar to the compounds of the invention, such as DCA, a known GAVE10 ligand, inhibit IL-6 production in macrophages, as well (FIG. 4). The method of the invention, therefore, may be used to treat any condition in which inhibiting TNFα and IL-6 is desirable, and, in particular, those diseases in which TNFα and IL-6 production is mediated by GAVE10. In a preferred embodiment, the method of the invention may be used to treat any condition associated with inflammation.

The term “treat,” as used here, means to prevent, lessen, or abolish a condition, or to otherwise alter it in a desirable manner. Hence, the term includes treating conditions prophylactically as well as treating established conditions.

“Conditions associated with inflammation” include any disease (that is, any condition that impairs normal functioning or is otherwise undesirable) in which inflammation plays a role, either as ultimate cause or proximal symptom.

Conditions associated with inflammation include rheumatoid and other forms of arthritis, such as bursitis, gout, and polymyalgia rheumatica; allergic rhinitis and sinusitis; asthma and bronchiectasis; ulcerative colitis and Crohn's disease; silicosis and other pneumoconiosis; cachexia; cholecystitis; psoriasis; multiple sclerosis; systemic lupus erythematosus; thyroiditis; atherosclerosis; juvenile diabetes; graft versus host disease; meningitis; contact hypersensistivity; anaphylactic states; chronic obstructive pulmonary disease; and any condition where the immune system reacts to an insult by causing leukocytes and/or plasma to collect at a site. In a preferred embodiment, the method of the invention is used to treat inflammation in which TNFα or IL-6 production is significant.

The method of the invention may further be used to activate GAVE10, and to treat those conditions in which doing so would be beneficial.

The inventors have shown previously (as they describe in co-pending application U.S. Ser. No. 10/491,376, the contents of which are incorporated by reference) that GAVE10 is derived from an intronless structural gene encoding about 330 amino acids, resulting in a polypeptide with a molecular weight of about 35 kD. The nucleic acid, comprising about 1586 base pairs (bp), including untranslated regions, is set forth in SEQ ID NO:1. The amino acid sequence is set forth in SEQ ID NO:2.

GAVE10 domains of interest include, but are not limited to, the transmembrane (TM) domains TM1 from about amino acid 15 to about 39; TM2 from about amino acid 50 to about 71; TM3 from about amino acid 84 to about 107; TM4 from about amino acid 124 to about 144; TM5 from about amino acid 159 to about 192; TM6 from about amino acid 227 to about 250; and TM7 from about amino acid 259 to about 282; intracellular (IC) domains IC1 from about amino acid 40 to about 49; IC2 from about amino acid 108 to about 123; IC3 from about amino acid 193 to about 226; and IC4 from about amino acid 283 to about 330; and extracellular (EC) domains EC1 from about amino acid 1 to about 4; EC2 from about amino acid 72 to about 83; EC3 from about amino acid 145 to about 158; and EC4 from about amino acid 251 to about 258 (all of the foregoing references to amino acids are to the amino acids of SEQ ID NO:2). In a related aspect, domains of interest also include, but are not limited to, consensus glycosylation sites, lipid binding sites and phosphorylation sites. Asparagine residues are located in the N-terminus and the EC2 and EC3 loops. Kinase phosphorylation sites such as serines are found in IC3 and the C-terminus. GAVE10 also possesses the ERY motif instead of the typical DRY motif downstream from TM3.

GAVE10 can be amplified using cDNA, mRNA or genomic DNA as a template and appropriate oligonucleotide primers according to standard PCR amplification techniques. For example, such primers can comprise, but are not limited to 5′-ATGACGCCCAACAGCACT-3′ (SEQ ID NO:3) and 5′-TTAGTTCAAGTCCAGGTC-3′ (SEQ ID NO:4). The nucleic acid so amplified can be cloned into an appropriate vector and characterized by DNA sequence analysis. Furthermore, oligonucleotides corresponding to GAVE10 nucleotide sequences can be prepared by standard synthetic techniques, e.g., using an automated DNA synthesizer.

GAVE10 fragments are also useful. Such a fragment can comprise, for example, a region encoding amino acid residues about 1 to about 14 of SEQ ID NO:2. The nucleotide sequence determined from the cloning of the human GAVE10 gene allows for the generation of probes and primers for identifying and/or cloning GAVE10 homologues in other cell types, for example, from other tissues, as well as GAVE10 homologues from other mammals. The probe/primer typically comprises substantially purified oligonucleotide. The oligonucleotide typically comprises a region of nucleotide sequence that hybridizes under stringent conditions to at least about 12, preferably about 25, more preferably about 50, 75, 100, 125, 150, 175, 200, 250, 300, 350 or 400 consecutive nucleotides of the sense or anti-sense sequence of SEQ ID NO:1 or of a naturally occurring mutant of SEQ ID NO:1. Probes based on the human GAVE10 nucleotide sequence can be used to detect transcripts or genomic sequences encoding the similar or identical proteins. The probe may comprise a label group attached thereto, e.g., a radioisotope, a fluorescent compound, an enzyme or an enzyme cofactor. Such probes can be used as part of a diagnostic test kit for identifying cells or tissues that do not express properly GAVE10 protein. That can be accomplished, for example, by measuring levels of a GAVE10-encoding nucleic acid in a sample of cells from a subject, detecting GAVE10 mRNA levels, or determining whether a genomic GAVE10 gene has been mutated or deleted.

A nucleic acid fragment encoding a biologically active portion of GAVE10 can be prepared by isolating a portion of SEQ ID NO:1 that encodes a polypeptide having a GAVE10 biological activity, expressing the encoded portion, and assessing its activity. For example, a nucleic acid fragment encoding a biologically active portion of GAVE10 includes a third intracellular loop domain (amino acid residues from about 202 to about 219 as set forth in SEQ ID NO:2).

An isolated nucleic acid molecule encoding a GAVE10 protein having a sequence that differs from that of SEQ ID NO:2 can be created by introducing one or more nucleotide substitutions, additions or deletions into the nucleotide sequence of SEQ ID NO:1 such that one or more amino acid substitutions, additions or deletions are introduced into the encoded protein.

Mutations can be introduced by standard techniques, such as site-directed mutagenesis and PCR-mediated mutagenesis. Preferably, conservative amino acid substitutions are made at one or more predicted non-essential amino acid residues. A “conservative amino acid substitution” is one in that the amino acid residue is replaced with an amino acid residue having a similar side chain. Families of amino acid residues having similar side chains are defined in the art. The families include amino acids with basic side chains (e.g., lysine, arginine and histidine), acidic side chains (e.g., aspartic acid and glutamic acid), uncharged polar side chains (e.g., glycine, asparagine, glutamine, serine, threonine, tyrosine and cysteine), nonpolar side chains (e.g., alanine, valine, leucine, isoleucine, proline, phenylalanine, methionine and tryptophan), beta-branched side chains (e.g., threonine, valine and isoleucine) and aromatic side chains (e.g., tyrosine, phenylalanine, tryptophan and histidine). Thus, a predicted nonessential amino acid residue in GAVE10 preferably is replaced with another amino acid residue from the same side chain family. Alternatively, mutations can be introduced randomly along all or part of a GAVE10 coding sequence, such as by saturation mutagenesis, and the resultant mutants can be screened for GAVE10 biological activity to identify mutants that retain activity. Following mutagenesis, the encoded protein can be expressed recombinantly and the activity of the protein can be determined.

Eosinophils mediate much of the airway dysfunction in allergy and asthma. Interleukin-5 (IL-5) is an eosinophil growth and activating cytokine. Studies have shown IL-5 to be necessary for tissue eosinophilia and for eosinophil-mediated tissue damage resulting in airway hyperresponsiveness (Chang et al., J Allergy Clin Immunol (1996) 98(5 pt 1):922-931 and Duez et al., Am J Respir Crit Care Med (2000) 161(11):200-206). IL-5 is made by T-helper-2 cells (Th2) following allergen (e.g. house dust mite antigen) exposure in atopic asthma.

RA is believed to result from accumulation of activated macrophages in the affected synovium. Interferon γ (IFNγ) is a T-helper-1 (Th1) cell-derived cytokine with numerous proinflammatory properties. It is the most potent macrophage activating cytokine and induces MHC class II gene transcription contributing to a dendritic cell-like phenotype. Lipopolysaccharide (LPS) is a component of gram-negative bacterial cell walls that elicits inflammatory responses, including tumor necrosis factor α (TNFα) release. The efficacy of intravenous anti-TNFα therapy in RA has been demonstrated in the clinic. COPD is thought also to result from macrophage accumulation in the lung, the macrophages produce neutrophil chemoattractants (e.g., IL-8: de Boer et al., J Pathol (2000) 190(5):619-626). Both macrophages and neutrophils release cathepsins that cause degradation of the alveolar wall. It is believed that lung epithelium can be an important source for inflammatory cell chemoattractants and other inflammatory cell-activating agents (see, for example, Thomas et al., J Virol (2000) 74(18):8425-8433; Lamkhioued et al., Am J Respir Crit Care Med (2000) 162(2 Pt. 1):723-732; and Sekiya et al., J Immunol (2000) 165(4):2205-2213).

Using a Northern blot assay, a GAVE10 mRNA transcript of approximately 1.8 kb was expressed in certain tissues. GAVE10, by RT-PCR, was shown to be expressed in THP-1 exposed to LPS. The receptor in transfected HEK293 cells also shows constitutive activation in the absence of agonist. Further, Northern blot results indicated that GAVE10 expression is not detected in brain, skeletal muscle or pancreas. In contrast, GAVE10 is expressed in placenta, liver and kidney, and weakly expressed in the heart. In a related aspect, TaqMan RT-PCR experiments were carried out to further evaluate GAVE10 expression. Results from a tissue panel indicated that GAVE10 showed expression in spleen, and also in kidney, heart, thymus and liver. GAVE10 expression also was elevated in activated vascular endothelial cells, activated macrophages such as by exposure to IFNγ and activated CD19 cells. GAVE10 expression was elevated in fibroblast-like synoviocytes activated by exposure to IL-1 or TNF. GAVE10 expression is elevated in synovial tissue from patients having rheumatoid arthritis or osteoarthritis.

The compounds of Formula I are preferably administered as pharmaceutically acceptable salts. Pharmaceutically acceptable acid salts are those of any suitable inorganic or organic acid. Suitable inorganic acids are, for example, hydrochloric, hydrobromic, sulfuric, and phosphoric acids. Suitable organic acids include carboxylic acids, such as, acetic, propionic, glycolic, lactic, pyruvic, malonic, succinic, fumaric, malic, tartaric, citric, cyclamic, ascorbic, maleic, hydroxymaleic, and dihydroxymaleic, benzoic, phenylacetic, 4-aminobenzoic, 4-hydroxybenzoic, anthranillic, cinnamic, salicyclic, 4-aminosalicyclic, 2-phenoxybenzoic, 2-acetoxybenzoic, and mandelic acid, sulfonic acids, such as, methanesulfonic, ethanesulfonic and β-hydroxyethanesulfonic acid. Salts of the compounds of Formula I formed with inorganic or organic bases are also included within the scope of this invention and include, for example, those of alkali metals, such as, sodium, potassium and lithium, alkaline earth metals, for example, calcium and magnesium, light metals of group IIIA, for example, aluminum, organic amines, such as, primary, secondary or tertiary amines, for example, cyclohexylamine, ethylamine, pyridine, methylaminoethanol and piperazine. The salts are prepared by conventional means, as for example, by treating a compound of Formula I with an appropriate acid or base.

The compounds of the invention may also be administered as prodrugs. As used here, the term “prodrug” refers to any compound that is converted into an active compound of the invention by metabolic processes within the body. There are various reasons why one might wish to administer a prodrug of the compounds of Formula I rather than the compound itself. Depending on the particular compound that one uses, a prodrug might have superior characteristics as far as solubility, absorption, stability, release, toxicity, and patient acceptability are concerned. It should be readily apparent to one of ordinary skill in the art how one can make a prodrug of any compound of the invention. There are many strategies for doing so. One can replace one or more of the oxygen atoms with hydrogen, for example. Such prodrugs are converted in vivo by enzymatic hydroxylation to the active compounds of the invention. Other prodrugs should be readily apparent to one of ordinary skill in the art.

The compounds of the invention may also be administered as solvates, that is, as compounds in physical association with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolable solvates. Exemplary solvates include ethanolates, methanolates, and the like. A “hydrate,” as used here, is a solvate wherein one or more solvent molecules is H₂O.

The compounds of the invention are preferably administered in association with a pharmaceutically acceptable carrier, for example, an adjuvant, diluent, coating and excipient. The compounds may be thus administered in any variety of suitable forms, such as by inhalation or topical, parenteral, rectal, or oral administration; preferably, they are administered orally. More specific routes of administration include intravenous, intramuscular, subcutaneous, intraocular, intrasynovial, colonical, peritoneal, and transepithelial, including transdermal, ophthalmic, sublingual, buccal, dermal, and ocular administration, and by nasal inhalation via insufflation.

The compounds of Formula I may be presented in forms permitting administration by the most suitable route. Such compositions may be prepared according to the customary methods, using one or more pharmaceutically acceptable adjuvants or excipients. The adjuvants comprise, among other things, diluents, sterile aqueous media and the various non-toxic organic solvents. The compositions may be presented in the form of tablets, pills, granules, powders, aqueous solutions or suspensions, injectable solutions, elixirs or syrups, and may contain one or more agents chosen from the grip comprising sweeteners such as sucrose, lactose, fructose, saccharin or aspartame, flavorings such as peppermint oil, oil of wintergreen, or cherry or orange flavorings, colorings, or stabilizers such as methyl- or propyl-paraben in order to obtain pharmaceutically acceptable preparations.

The choice of vehicle and the content of active substance in the vehicle are generally determined in accordance with the solubility and chemical properties of the product, the particular mode of administration and the provisions to be observed in pharmaceutical practice. For example, excipients such as lactose, sodium citrate, calcium carbonate, dicalcium phosphate and disintegrating agents such as starch, alginic acids and certain complex silica gels combined with lubricants such as magnesium stearate, sodium lauryl sulfate and talc may be used for preparing tablets, troches, pills, capsules and the like. To prepare a capsule, it is advantageous to use lactose and liquid carrier, such as high molecular weight polyethylene glycols. Various other materials may be present as coatings or to otherwise modify the physical form of the dosage unit. For instance, tablets, pills, or capsules may be coated with shellac, sugar or both. When aqueous suspensions are used they may contain emulsifying agents or agents which facilitate suspension. Diluents such as sucrose, ethanol, polyols such as polyethylene glycol, propylene glycol and glycerol, and chloroform or mixtures thereof may also be used. In addition, the active compound may be incorporated into sustained-release preparations and formulations.

For oral administration, the active compound may be administered with, for example, an inert diluent or with an assimilable edible carrier, or it may be enclosed in hard or soft shell gelatin capsules, or it may be compressed into tablets, or it may be incorporated directly with the food of the diet, or may be incorporated with excipient and used in the form of ingestible tablets, buccal tablets, troches, capsules, elixirs, suspensions, syrups, wafers, and the like.

For parenteral administration, emulsions, suspensions or solutions of the compounds according to the invention in vegetable oil, for example sesame oil, groundnut oil or olive oil, or aqueous-organic solutions such as water and propylene glycol, injectable organic esters such as ethyl oleate, as well as sterile aqueous solutions of the pharmaceutically acceptable salts, are used. The injectable forms must be fluid to the extent that it can be easily syringed, and proper fluidity can be maintained, for example, by the use of a coatings such as lecithin, by the maintenance of the required particle size in the case of dispersion and by the use of surfactants. Prolonged absorption of the injectable compositions can be brought about by use of agents delaying absorption, for example, aluminum monostearate and gelatin. The solutions of the salts of the products according to the invention are especially useful for administration by intramuscular or subcutaneous injection. Solutions of the active compound as a free base or pharmacologically acceptable salt can be prepared in water suitably mixed with a surfactant such as hydroxypropyl-cellulose. Dispersion can also be prepared in glycerol, liquid polyethylene glycols, and mixtures thereof and in oils. The aqueous solutions, also comprising solutions of the salts in pure distilled water, may be used for intravenous administration with the proviso that their pH is suitably adjusted, that they are judiciously buffered and rendered isotonic with a sufficient quantity of glucose or sodium chloride and that they are sterilized by heating, irradiation, microfiltration, and/or by various antibacterial and antifungal agents, for example, parabens, chlorobutanol, phenol, sorbic acid, thimerosal, and the like.

Sterile injectable solutions are prepared by incorporating the active compound in the required amount in the appropriate solvent with various of the other ingredients enumerated above, as required, followed by filtered sterilization. Generally, dispersions are prepared by incorporating the various sterilized active ingredient into a sterile vehicle which contains the basic dispersion medium and the required other ingredients from those enumerated above. In the case of sterile powders for the preparation of sterile injectable solutions, the preferred methods of preparation are vacuum drying and the freeze drying technique which yield a powder of the active ingredient plus any additional desired ingredient from previously sterile-filtered solution thereof.

Topical administration, gels (water or alcohol based), creams or ointments containing compounds of the invention may be used. Compounds of the invention may be also incorporated in a gel or matrix base for application in a patch, which would allow a controlled release of compound through transdermal barrier.

For administration by inhalation, compounds of the invention may be dissolved or suspended in a suitable carrier for use in a nebulizer or a suspension or solution aerosol, or may be absorbed or adsorbed onto a suitable solid carrier for use in a dry powder inhaler.

Solid compositions for rectal administration include suppositories formulated in accordance with known methods and containing at least one compound of Formula I.

Compositions according to the invention may also be formulated in a manner which resists rapid clearance from the vascular (arterial or venous) wall by convection and/or diffusion, thereby increasing the residence time of the viral particles at the desired site of action. A periadventitial depot comprising a compound according to the invention may be used for sustained release. One such useful depot for administering a compound according to the invention may be a copolymer matrix, such as ethylene-vinyl acetate, or a polyvinyl alcohol gel surrounded by a Silastic shell. Alternatively, a compound according to the invention may be delivered locally from a silicone polymer implanted in the adventitia.

An alternative approach for minimizing washout of a compound according to the invention during percutaneous, transvascular delivery comprises the use of nondiffusible, drug-eluting microparticles. The microparticles may be comprised of a variety of synthetic polymers, such as polylactide for example, or natural substances, including proteins or polysaccharides. Such microparticles enable strategic manipulation of variables including total dose of drug and kinetics of its release. Microparticles can be injected efficiently into the arterial or venous wall through a porous balloon catheter or a balloon over stent, and are retained in the vascular wall and the periadventitial tissue for at least about two weeks. Formulations and methodologies for local, intravascular site-specific delivery of therapeutic agents are discussed in Reissen et al. (J. Am. Coll. Cardiol, 1994; 23: 1234-1244), the entire contents of which are hereby incorporated by reference.

A composition according to the invention may also comprise a hydrogel which is prepared from any biocompatible or non-cytotoxic (homo or hetero) polymer, such as a hydrophilic polyacrylic acid polymer that can act as a drug absorbing sponge. Such polymers have been described, for example, in application WO93/08845, the entire contents of which are hereby incorporated by reference. Certain of them, such as, in particular, those obtained from ethylene and/or propylene oxide are commercially available.

The percentage of active ingredient in the compositions of the invention may be varied, but there should be a sufficient proportion such that a suitable dosage is obtained. Several unit dosage forms may be administered at about the same time.

The compositions of the invention are administered to a subject (meaning a human, preferably, but including any other mammal, as well) in an effective amount, that is, in an amount effective to treat inflammation. The precise dose depends upon the desired therapeutic effect, the route of administration, the duration of the treatment, and the condition of the patient. In the adult human, the doses are generally from about 0.001 to about 50 (preferably about 0.001 to about 5) mg/kg body weight per day by inhalation, from about 0.01 to about 100 (preferably 0.1 to 70, and more preferably 0.5 to 10) mg/kg body weight per day by oral administration, and from about 0.001 to about 10 (preferably 0.01 to 10) mg/kg body weight per day by intravenous administration. In each particular case, the doses are determined in accordance with the factors distinctive to the patient to be treated, such as age, weight, general state of health and other characteristics which can influence the efficacy of the compound according to the invention.

The compositions of the invention may be administered as frequently as necessary in order to obtain the desired therapeutic effect. Some patients may respond rapidly to a higher or lower dose and may find much weaker maintenance doses adequate. For other patients, it may be necessary to have long-term treatments at the rate of 1 to 4 doses per day, in accordance with the physiological requirements of each particular patient. Generally, the compositions may be administered orally 1 to 4 times per day. Of course, for other patients, it will be necessary to prescribe not more than one or two doses per day.

### EXAMPLES

The invention is illustrated further by the following examples.

### Example 1

### Cloning hGAVE10 cDNA

Human genome banks were mined for GPCR motifs. A human genomic DNA, AC021016.3, gi7630969, was selected (the genomic DNA, and fragments thereof, could be used as probe in Northern blots). PCR screening was performed on a pool of human kidney, thymus and placenta cDNA libraries. Primers for PCR were designed using the following sequences:

Forward: 5′-CAGGACCAAGATGACGCCCA-3′ (SEQ ID NO:6)

Nested Forward: 5′-CGAAGCTTCAGGACCAAGATGAGC-3′ (SEQ ID NO:7)

The nested forward primer contains a HindIII restriction enzyme site followed by a Kozak sequence. The nested reverse primer contains an XhoI restriction enzyme site. PCR was carried out in a Biometra Trio-Thermoblock thermocycler, using Pfu DNA polymerase (Stratagene) that was added to the PCR reaction following addition of template cDNA, primers, Pfu buffer and dNTP. The 50 μl reaction contains: 5 μl of l0X Pfu DNA buffer, 2 μl (2500 units/ml) of Pfu DNA polymerase, 1.0 μl of NTP mixture (containing 10 mM of each nucleotide); 2.0 μl of forward primer (10 mM); 2.0 μl of reverse primer (10 mM); 5 μl cDNA template and 33 μl sterile water. The following cycles were used in the thermocycler: 94° C. for 2 minutes, followed by 30 cycles of 94° C. for 45 seconds, 58° C. for 45 seconds, 72° C. for 3 minutes, 72° C. for 10 minutes; and cooling down at 4° C.

Following PCR, 3 μl of dNTP (10 mM of each nucleotide) Clontech Catalog No. 7404-i and 1 μl (5 units) of Taq DNA Polymerase (Qiagen, Catalog No. 201223) were added to the PCR product and the mixture was incubated at 72° C. for 10 minutes. The PCR product then was run on a 1% agarose gel. About a 1 kilobase band containing the desired fragment was cut from the gel and purified using the Qiaquick Gel Extraction Kit using the protocol provided by the manufacturer (Qiagen, Catalog No. 28704). The purified PCR product then was subcloned into a pCR2.1 vector (Invitrogen, Catalog Nos. K2000-01/40/J10 and K2030-01/40/J10). To subclone the PCR product into the pCR2.I vector, a ligation reaction was prepared using an Invitrogen TA cloning vector kit. The ligation reaction contained: 5 μl sterile water; 1 μl Invitrogen 2× ligation buffer; 2 μl pCR2.1 vector (25 ng/μl); 4 μl PCR product DNA (10 ng); 4 μl (5×) dilution buffer; and 1 μl T4 DNA ligase (5 units). The reaction was incubated for 18 hours at 14° C. E. coli cells were transformed with the ligation reaction by mixing 2 μl of the ligation reaction mixture with 200 μl of INVα F′ competent E. coli cells (Invitrogen Catalog No. C658-00), incubation on ice for 30 minutes, heat shock at 37° C. for 45 seconds and incubation on ice for 2 minutes followed by addition of 800 μl of LB. The cells then were incubated overnight at 37° C. with agitation in a bacterial shaker/incubator (air was re-circulated). Following the overnight incubation, 200 μl of the transformation reaction mixture was plated onto LB agar plates containing 100 μg/ml ampicillin and incubated overnight at 37° C.

Following the incubation, colonies were picked and each individual colony was grown in a separate tube overnight in 500 μl of LB containing 100 μg/ml ampicillin in a shaker/incubator. To screen colonies by PCR, the following reaction was used: 41.5 μl of a colony in LB; 5 μl Taq buffer (10×); 1.0 μl dNTP (10 mM of each nucleotide); 1.0 μl forward primer (10 mM); 1.0 μl reverse primer (10 mM); and 0.5 μl Taq DNA polymerase (5 units/μl).

The reaction was incubated in a thermocycler using the following cycles: 94° C. for 2 minutes, 94° C. for 30 seconds, 55° C. for 30 seconds, 72° C. for 1 minute and 72° C. for 10 minutes, followed by cooling down at 4° C.

To check the results of the PCR reaction, 5 μl of the PCR reaction was run on 1% TAB agarose gel. Positive clones showed an insert of about 1 kb. Positive clones were grown in 5 ml LB+100 μg/ml ampicillin overnight at 37° C. in a bacterial shaker/incubator. The plasmid was purified using a Qiagen DNA purification column as instructed in the manufacturer protocol (Qiagen Catalog No. 12143). The positive clones then were sequenced using a T7 forward primer (5′-GGCTCCCAACTTCTCTTC-3′) (SEQ ID NO:8) and an M13 reverse primer (5′-GGGCAGTGGCCAGCACGC-3′) (SEQ ID NO:9). DNA sequencing identified isolation of a cDNA having the DNA sequence presented in FIG. 1 (SEQ ID NO:1) and the amino acid sequence presented in FIG. 2 (SEQ ID NO:2).

### Example 2

### Generation of Mammalian Cells Overexpressing hGAVE10

To provide significant quantities of hGAVE10 for further experiments, the cDNA encoding hGAVE10 was cloned into an expression vector and transfected into mammalian cells, such as 293 cells.

To generate mammalian cells overexpressing hGAVE10, mammalian cells were plated in a six-well 35 mm tissue culture plate (3×10⁵ mammalian cells per well (ATCC Catalog No. CRL-1573)) in 2 ml of DMEM media (Gibco/BRL, Catalog No. 11765-054) in the presence of 10% fetal bovine serum (Gibco/BRL Catalog No. 1600-044). The cells then were incubated at 37° C. in a CO₂ incubator until the cells were 50-80% confluent. The cloned cDNA nucleic acid sequence of hGAVE10 was inserted using the procedure described above in a pcDNA 3.1 cloning vector (Invitrogen, Catalog No. V790-20). Two μg of the DNA were diluted into 100 μl of serum-free μl 2 HAM media. Separately, 25 μl of Lipofectamine Reagent (Life Technologies, Catalog No. 18324-020) was diluted into 100 μl of serum-free F12 HAM media. The DNA solution and the Lipofectamine solution then were mixed gently and incubated at room temperature for 45 minutes to allow for the formation of DNA-lipid complexes. The cells were rinsed once with 2 ml of serum-free F12 HAM media. For each transfection (six transfections in a six-well plate), 0.8 ml of serum-free F12 HAM media were added to the solution containing the DNA-lipid complexes (0.2 ml total volume) and mixed gently. The resulting mixture (hereinafter the “transfection mixture”) then was overlaid (0.8 ml+0.2 ml) onto the rinsed cells. No anti-bacterial reagents were added. The cells then were incubated with the lipid-DNA complexes for 16 hours at 37° C. in a CO₂ incubator to allow for transfection.

After the completion of the incubation period, 1 ml of F12 HAM media containing 10% fetal bovine serum was overlaid onto the cells without first removing the transfection mixture. At 18 hours after transfection, the media overlaying the cells was aspirated. Cells then were washed with PBS, pH 2-4 (Gibco/BRL Catalog No. 10010-023) and the PBS was replaced with F12 HAM media containing 5% serum (“selective media”). At 72 hours after transfection, the cells were diluted ten-fold into the selective medium containing the antibacterial agent genetecin at 400 μg/ml (Life Technologies, Catalog No. 11811).

### Example 3

### Agonist Assay

To screen for agonists of human GAVE10, hGAVE10 was coupled artificially to a G_{q} mechanism. Activation of the G_{q} mechanism stimulates the release of Ca²⁺ from sarcoplasmic reticulum vesicles within the cell. The Ca²⁺ was released into the cytoplasm where it can be detected using Ca²⁺ chelating dyes. A Fluorometric Imaging Plate Reader (i.e., a FLIPR® apparatus, Molecular Devices) was used to monitor any resulting changes in fluorescence. The activity of an agonist was reflected by any increase in fluorescence.

CHO-KI cells expressing hGAVE10 were pre-engineered to express an indiscriminate form of G_{q} protein (G₀₁₆). To prepare such cells, G_{α16}-coupled CHO cells were obtained commercially (Molecular Devices LIVEWARE™ cells, Catalog No. RD-HGAI6) and the protocol in Example 2 followed to facilitate expression of hGAVE10 in those cells. The cells were maintained in log phase of growth at 37° C. and 5% CO₂ in F12 Ham's media (Gibco/BRL, Catalog No. 11765-054) containing 10% fetal bovine serum, 100 IU/ml penicillin (Gibco/BRL, Catalog No. 15140-148), 100 μg/ml streptomycin (Catalog No. 15140-148, Gibco/BRL), 400 μg/ml genetecin (G418) (Gibco/BRL, Catalog No. 10131-035) and 200 μg/ml zeocin (Invitrogen, Catalog No. R250-05). One day prior to an assay, 12,500 cells/well of the CHO-K1 cells were plated onto 384-well clear-bottomed assay plates with a well volume of 50 μl (Greiner/Marsh, Catalog No. N58102) using a 96/384 Multidrop device (Labsystems, Type 832). The cells were incubated at 37° C. in a humidified 5% CO₂ incubator (Form a Scientific CO₂ water-jacketed incubator Model 3110).

The following stock solutions were prepared: a 1 M stock solution of Hepes (pH 7.5) (Gibco/BRL, Catalog No. 15630-080); a 250 mM stock solution of probenicid (Sigma, Catalog No. P8761) made in 1 N NaOH; and a 1 mM stock solution of Fluo 4-AM Dye (Molecular Probes, Catalog No. FI 4202) made in DMSO (Sigma D2650). Reaction buffer was prepared with 1000 ml Hank's balanced salt solution (Fisher/Mediatech, Catalog No. MT21023), 20 ml of the 1 M Hepes stock solution and 10 ml of the 250 mM probenicid stock solution. To prepare the loading buffer, 1.6 ml of the 1 mM Fluo 4-AM Dye stock solution was mixed with 0.32 ml of pluronic acid (Molecular Probes, Catalog No. P6866) and then mixed with 400 ml of the above reaction buffer and 4 ml of fetal bovine serum.

One hour prior to the assay, 50 μl of freshly-prepared loading buffer was added to each well of the 384-well plate using a 96/384 Multidrop device. The cells were incubated at 37° C. in a humidified incubator to maximize dye uptake. Immediately prior to the assay, the cells were washed 2 times with 90 μl of reaction buffer using a 384 EMBLA Cell Washer (Skatron; Model No. 12386) with the aspiration head set at least 10 mm above the plate bottom, leaving 45 μl of buffer per well.

The CCD camera (Princeton Instruments) of the FLIPR® II (Molecular Devices) instrument was set at an f-stop of 2.0 and an exposure of 0.4 seconds. The camera was used to monitor the cell plates for accuracy of dye loading. A compound library containing possible agonists was tested at a concentration of 10 pM in physiological salt buffer. Changes in fluorescence were measured for 10 seconds prior to compound addition. After the addition of the compound, fluorescence was measured every second for the first minute followed by exposures taken every six seconds for a total experimental analysis time of three minutes. Five μl aliquots of the 100 μM stock compound were added after the tenth scan, giving a final compound concentration on the cells of 10 μM. The maximum fluorescence changes for the first 80 scans were recorded as a measure of agonist activity and compared to the maximum fluorescence change induced by 10 μM ATP (Sigma A9062).

### Example 4

### Antagonist Assay

To screen for antagonists of human GAVE10, hGAVE10 was coupled artificially to a G_{q} mechanism. As in Example 3, a FLIPR® apparatus was used to monitor any resulting changes in fluorescence. The activity of an antagonist was reflected by any decrease in fluorescence.

CHO-K1 cells expressing hGAVE10 were pre-engineered to express an indiscriminate form of G_{q} protein (G_{α16}), as described in Example 3. The cells were maintained in log phase of growth at 37° C. and 5% CO₂ in F12 HAM media (Gibco/BRL, Catalog No. 11765-054) containing 10% fetal bovine serum, 100 IU/ml penicillin (Gibco/BRL, Catalog No. 15140-148), 100 μg/ml streptomycin (Catalog No. 15140-148, Gibco/BRL), 400 μg/ml genetecin (G418) (Gibco/BRL, Catalog No. 10131-035) and 200 μg/ml zeocin (Invitrogen, Catalog No. R250-05). One day prior to the assay, 12,500 cells/well of the CHO-K1 cells were plated onto 384-well black/clear bottomed assay plates with a well volume of 50 μl (Greiner/Marsh, Catalog No. N58102) using a 96/384 Multidrop device. The cells were allowed to incubate at 37° C. in humidified 5% CO₂.

The following stock solutions were prepared: a 1 M stock solution of Hepes (pH 7.5) (Gibco/BRL, Catalog No. 15630-080); a 250 mM stock solution of probenicid (Sigma, Catalog No. P8761) made in 1 N NaOH; a 1 mM stock solution of Fluo 4-AM Dye (Molecular Probes, Catalog No. F 14202) made in DMSO (Sigma D2650); and a stock solution of ligand or antagonist. Reaction buffer was prepared with 1000 ml Hank's balanced salt solution (Fisher/Mediatech, Catalog No. MT21023), 20 ml of the 1 M Hepes stock solution, 10 ml of the 250 mM probenicid stock solution and 1 mM CaCl₂. To prepare the loading buffer, 80 μl of the 1 mM Fluo 4-AM Dye stock solution was mixed with 16 μl of pluronic acid (Molecular Probes, Catalog No. P6866) and then mixed with 20 ml of the above reaction buffer and 0.2 ml of fetal bovine serum.

Thirty minutes prior to the assay, 30 μl of freshly-prepared loading buffer was added to each well of the 384-well plate using a 96/384 Multidrop device. The cells were incubated at 37° C. in a humidified CO₂ incubator to maximize dye uptake. Immediately prior to the assay, the cells were washed 3 times with 100 μl of reaction buffer using a 384 EMBLA Cell Washer with the aspiration head set at least 40 mm above the plate bottom, leaving 45 μl of buffer per well.

Five μl of the 100 μM stock antagonist compound were added to the cells using a Platemate-384 pipettor (Matrix). The compound concentration during the incubation step was approximately 10 μM. The cells were placed on the FLIPR® II and plate fluorescence was measured every second for the first minute followed by exposures taken every six seconds for a total experimental analysis time of three minutes. Antagonist or ligand (10 μM) was added after the tenth scan. After each addition, the 384 tips were washed 10 times with 20 μl of 0.01% DMSO in water.

### Example 5

### Receptor Binding Assay

To prepare membrane fractions containing hGAVE10 receptor, CHO cell lines overexpressing hGAVE10 were harvested by incubation in phosphate-buffered saline (10 ml) containing 1 mM EDTA. The cells were washed further 3 times in phosphate-buffered saline containing 1 mM EDTA (10 ml) prior to resuspension in 5 ml of Buffer A (50 mM Tris-HCl (pH 7.8) (Sigma T6791), 5 mM MgCl₂ (Sigma M8266) and 1 mM EGTA (Sigma 0396).

The cells then were disrupted with a tissue homogenizer (Polytron, Kinemetica, Model PT 10/35) for 1 minute. The resulting homogenate was centrifuged in a Sorvall Instruments RC3B refrigerated centrifuge at 49,000×g at 4° C. for 20 minutes. The resulting pellet was resuspended in 25 ml of Buffer A and the centrifugation step was repeated three times. Following the final centrifugation, the pellet again was resuspended in 5 ml of Buffer A, aliquoted and stored at −70° C.

A receptor binding assay using the membrane fraction and radiolabeled ligand or agonist as a tracer was performed. The assay was performed in a 96-well plate (Beckman Instruments). The binding reaction consists of 18 μg of the CHO cell preparation in the presence of radioactive ligand or agonist (0.01 nM-25 nM) in a final volume of 0.2 ml of Buffer A containing 0.1% bovine serum albumin (Sigma, Catalog No. 34287) (see Im et al., J Biol Chem (2000) 275(19):14281-14286). The reaction was incubated for 1 hour at room temperature. The reaction was terminated by filtration through Whatman GF/C filters on a multichannel harvester (Brandell) that was pretreated with 0.3% polyethyleneimine (Sigma, Catalog No. P3143) and 0.1% bovine serum albumin (BSA) for 1 hour. The mixture was applied to the filter and incubated for one hour. The filters were washed 6 times with 1 ml of ice cold 50 mM Tris-HCl, pH 7.6. Specific binding was calculated based on the difference between total binding and non-specific binding (background) for each tracer concentration by measuring the radioactivity. Eight to 16 concentration data points were obtained to determine the binding of ligand to the receptor achieved in an equilibrium state between the ligand and receptor (equilibrium binding parameters) and the amount of nonradioactive ligand or agonist needed to compete for the binding of radioactive ligand or agonist on the receptor (competition binding values). Inhibition curves were prepared to determine the concentration required to achieve a 50% inhibition of binding (IC₅₀).

### Example 6

### Northern Blot Analysis

Northern blot analysis was performed on total RNA or poly A⁺ RNA derived from several human tissue samples to determine whether the tissues express hGAVE10. The probe used was P³²-labeled hGAVE10 cDNA or portions thereof.

Preparation of the Probe

P³²-labeled hGAVE10 cDNA was prepared as follows. Twenty-five ng of hGAVE10 cDNA prepared as described above was resuspended to 45 μl of 10 mM Tris-HCl, pH 7.5; 1 mM EDTA in a microfuge tube and heated at 95° C. for 5 minutes. The tube then was chilled on ice for another 5 minutes. Following chilling, the mixture contained in the tube was resuspended with the 45 μl GAVE10 cDNA and buffer as described above and mixed with RTS Rad Prime Mix (supplied with the RTS Rad Prime DNA-labeling System) (Life Technologies, Catalog No. 10387-017). Five μl of P³²-labeled α-dCTP, specific activity 3000 Ci/mM, (Amersham, AA0005), were added while mixing gently but thoroughly. The resulting mixture was incubated at 37° C. for 10 minutes. Incubation was stopped by the addition of 5 μl of 0.2 M EDTA, pH 8.0. Incorporation of the radioactive α-dCTP into the hGAVE10 cDNA was evaluated by taking a 5 μl aliquot of the mixture and counting the radioactivity.

RNA Extraction

Cells of interest were lysed directly in a culture dish by adding 1 ml of Trizol Reagent (Life Technologies, Catalog No. 15596). The cell lysate then was passed through a pipette several times to homogenize the lysate (cell lysate subsequently was transferred to a tube). Following homogenization, the lysate was incubated for 5 minutes at 30° C. to permit the complete dissociation of nucleoprotein complexes. Following incubation, 0.2 ml of chloroform (Sigma, Catalog No. C5312) per 1 ml of Trizol Reagent were added to the lysate and the tube was shaken vigorously for 15 seconds. The lysate then was incubated at 30° C. for 3 minutes. Following incubation, the lysate was centrifuged at 12,000×g for 15 minutes at 4° C. The resulting aqueous phase was transferred to a fresh tube and 0.5 ml of isopropyl alcohol per 1 ml of Trizol Reagent were added. The aqueous phase sample then was incubated at 30° C. for 10 minutes and centrifuged at 12,000×g for 10 minutes at 4° C. Following centrifugation, the supernatant was removed and the remaining RNA pellet was rinsed with 70% ethanol. The rinsed sample then was centrifuged at 7500×g for 10 minutes at 4° C. and the resulting supernatant was discarded. The remaining RNA pellet then was dried and resuspended in RNase-free water (Life Technologies, Catalog No. 10977-015). Either total RNA, for example the samples from peripheral tissues, or poly A⁺ RNA, such as the samples of various brain regions, can be used in the Northern or Taqman (described below) experiments. Known standards, such as human brain actin of Perkin-Elmer, can be purchased.

Gel ElectroPhoresis

An agarose gel was prepared by melting 2 g of agarose (Sigma, Catalog No. A0169) in water, 5× formaldehyde gel-running buffer (see below for description) and 2.2 M formaldehyde (Sigma, Catalog No. P82031).

Samples for gel electrophoresis were prepared as follows:

| | |
|---|---|
| RNA | 4.5 μl (5 μg total) |
| 5X formaldehyde gel-running buffer | 2.0 μl |
| formaldehyde | 3.5 μl |
| formamide (Sigma, Catalog No. F9037) | 10.0 μl |

Formaldehyde gel-running buffer (5×) was 0.1 M 3-(N-morpholino) propanesulfonic acid (MOPS) (pH 7.0) (Sigma, Catalog No. M5162); 40 mM sodium acetate (Sigma, Catalog No. S7670); and 5 mM EDTA (pH 8.0) (Sigma, Catalog No. E7889).

The samples were incubated for 15 minutes at 65° C. and then chilled on ice. After chilling, the samples were centrifuged for 5 seconds. Two μl of formaldehyde gel-loading buffer; 50% glycerol (Sigma, Catalog No. G5516); l mM EDTA (pH 8.0); 0.25% bromophenol blue (Sigma, Catalog No. 18046); 0.25% xylene cyanol FF (Sigma, Catalog No. 335940) then were added to the sample.

Table 1 lists the sources of some of the RNA's used in some of the experiments.

TABLE 1

| Human Total RNA | Clontech Cat. No. |
|---|---|
| Human brain, whole | 64020-1 |
| Human Heart | 64025-1 |
| Human Kidney | 64030-1 |
| Human Liver | 64022-1 |
| Human Lung | 64023-1 |
| Human Pancreas | 64031-1 |
| Human Skeletal Muscle | 64033-1 |
| Human Small Intestine | 64039-1 |
| Human Spleen | 64034-1 |
| Human Stomach | 64090-1 |
| Human Thymus | 64028-1 |

The gel was pre-run for 5 minutes at 5 V/cm. Following the pre-run; the samples were loaded onto the gel. The gel then was run at 4 V/cm while submerged in IX formaldehyde gel-running buffer. The buffer was changed at 2 hours into the run.

Transfer of RNA from Gel to Nitrocellulose

The gel was stained with ethidium bromide (Sigma, Catalog No. El 385) (0.5 μg/ml in 0.1 M ammonium acetate (Sigma, Catalog No. 09689)) for 30 minutes to insure that RNA was not degraded. The RNA then was transferred from the agarose gel to a nitrocellulose filter (Schleicher & Schuell Inc., Catalog No. 74330-026) using the protocol described in Sambrook et al., eds. (in Molecular Cloning: A Laboratory Manual, volume 1, pp. 7.46-7.51, Cold Spring Harbor Laboratory Press (1989)).

Hybridization of P³²-labeled cDNA

Clontech ExpressHyb hybridization solution (Clontech, Catalog No. 8015-1) was incubated at 68° C. for 2 hours. Following incubation, 15 ml of the warmed hybridization solution was poured onto a multiple tissue sample Northern (MTN) membrane. The MTN membrane was left soaking in the hybridization solution at 68° C. while shaking. After 1 hour elapsed, the hGAVE10 cDNA probe, that had been previously denatured by boiling at 95° C. for 5 minutes, was added at a concentration of 106 counts/ml. The incubation of the hybridization solution covering the gel at 68° C. then was continued for 2 hours while shaking.

The MTN membrane then was removed from the Clontech ExpressHyb hybridization solution and washed 3 consecutive times with Clontech Wash Solution 1 (2×SSC; 0.05% SDS) by dipping the membrane into 15 ml of solution while shaking at room temperature for 40 minutes, respectively, with solution changes every 40 minutes. Clontech Wash Solution 2 (0.1×SSC; 0.1% SDS) then was warmed at 55° C. for 1 hour. The membrane then was washed 3 consecutive times with Clontech Wash Solution 2 (0.1×SSC; 0.1% SDS) by dipping the membrane into 15 ml of solution while shaking at 55° C. temperature for 60 minutes. The wash solution was changed every 15 minutes.

Development

The membrane was exposed to Kodak X-OMAT AR (Kodak, Catalog No. 1651579) film overnight at −70° C. and developed by standard methods. A number of different tissues were screened and a unique mRNA of about 2.3 kb was found in selected tissues, such as, spleen and lung.

### Example 7

### PCR Assay

TaqMan® or real time RT-PCR detects messenger RNA in samples. The assay exploits the 5′ nuclease activity of AmpliTaq Gold® DNA polymerase to cleave a TaqMan® probe during PCR. The TaqMan® probe contains a reporter dye for example, 6-FAM (6-carboxyfluorescein) at the 5′-end of the probe and a quencher dye (for example, TAMRA (6-carboxy-N, N, N′,N′-tetramethylrhodamine) at the 3′-end of the probe. TaqMan® probes were designed specifically to hybridize with the target cDNA of interest between the forward and the reverse primer sites. When the probe was intact, the 3′-end quencher dye suppresses the fluorescence of the 5′-end reporter dye. During PCR, the 5′→3′ activity of the AmpliTaq Gold® DNA polymerase results in the cleavage of the probe between the 5′-end reporter dye and the 3′-end quencher dye resulting in the displacement of the reporter dye. Once displaced, the fluorescence of the reporter dye no longer is suppressed by the quencher dye. Thus, the accumulation of PCR products made from the targeted cDNA template was detected by monitoring the increase in fluorescence of the reporter dye.

An ABI Prism Sequence detector system from Perkin Elmer Applied Biosystems (Model No. ABI7700) was used to monitor the increase of the reporter fluorescence during PCR. The reporter signal was normalized to the emission of a passive reference.

Preparation of cDNA Template

Total RNA and poly A⁺ RNA from several tissues can be purchased commercially, for example, from Clontech (see Table 1 above and Table 2 below).

TABLE 2

| RNA Sample | Clontech Catalog No. |
|---|---|
| Human Brain, whole | 6516-1 |
| Human Brain, amygdala | 6574-1 |
| Human Brain, caudate | 6575-1 |
| nucleus | |
| Human Brain, cerebellum | 6543-1 |
| Human Brain, corpus | 6577-1 |
| callosum | |
| Human Brain, hippocampus | 6578-1 |
| Human Brain, substantia | 6580-1 |
| nigra | |
| Human Brain thalamus | 6582-1 |
| Human Fetal Brain | 6525-1 |

Five μg of total RNA was mixed with 2 μl (50 ng/μl) of random hexamer primers (Life Technologies, Catalog No. 18090) for a total reaction volume of 7 μl. The resulting mixture was heated at 70° C. for 10 minutes and quickly chilled on ice. The following then were added to the mixture: 4 μl of 5× first strand buffer, 2 μl of 0.1 mM DTT, 1 μl of 10 mM dNTP and 1 μl of water. The mixture was mixed gently and incubated at 37° C. for 2 minutes. Following the incubation, 5 μl of Superscript RT-PCR reverse transcriptase (Life Technologies, Catalog No. 18090) was added. The mixture then was incubated at 37° C. for 60 minutes. The reaction was stopped by the addition of 1 μl of 2.5 mM EDTA. The mixture then was incubated for 65° C. for 10 minutes.

PCR and TagMan® Assay

The PCR and TaqMan® Assay were performed in a 96-well plate MicroAmp optical tube (Perkin Elmer, Catalog No. N801-0933). A reaction mixture comprising 25 μl of TaqMan®PCR Mixture (Perkin Elmer, Catalog No. N808-0230), 1 μl forward primer (5′-TGCTCTTTGCCAGTCTGCC-3′) (SEQ ID NO:10), 1 μl of reverse primer (5′-AAGATAGCCTGGGAGCTGCA-3′) (SEQ ID NO:11), 1 μl of TaqMan® probe (5′-TGGAACCACTGGACCCCTGGTGC-3′) (SEQ ID NO:12), 1 μl cDNA and 21 μl of water were placed into each well. TaqMan® samples were created in duplicate for each tissue sample at the following cDNA template concentrations: 5, 2, 1, 0.5, 0.25, 0.125, 0.0625 ng/μl (the template cDNA concentration was a final concentration). The plate then was sealed with MicroAmp optical 8-strip caps (Perkin Elmer, Catalog No. N801-0935).

A standard curve was performed in duplicate using the human β actin gene (Perkin Elmer, Catalog No. 401846). For each cDNA template concentration of the standard curve, a number of amplified molecules were obtained. Having a standard curve amplification of a known gene allows for quantification of cDNA molecules amplified for each unknown target gene and normalization with an internal control. Results from the above TaqMan® reactions were expressed relative to a tissue of arbitrary choice as fold regulation (for instance, value of GAVE10 expression in the spleen divided by the value of GAVE10 expression in the brain). Alternatively, a different tissue of known reactivity can be used as the frame of reference, such as actin. High levels of GAVE10 mRNA were observed.

### Example 8

### Identification of Inverse Agonist and Agonist Using [³⁵S]GTPγS

Membranes comprising the constitutively active receptors were prepared by first aspirating the media from a confluent monolayer of eukaryotic cells expressing GAVE10 (cells may be in a flask or multi-welled plate), followed by rinsing with 10 ml of cold PBS and further aspiration. Five ml of a buffer containing 20 mM HEPES and 10 mM EDTA, pH 7.4 were added to scrape the cells from the substratum. The cellular material was transferred into 50 ml centrifuge tubes (centrifuge at 20,000 rpm for 17 minutes at 4° C.). Thereafter the supernatant was aspirated and the resulting pellet was resuspended in 30 ml of a buffer containing 20 mM HEPES and 0.1 mM EDTA, pH 7.4, which was followed by centrifugation as above. The supernatant then was aspirated and the resulting pellet was resuspended in a buffer containing 20 mM HEPES, 100 mM NaCl and 10 mM MgCl₂ (binding buffer). The suspension then was homogenized using a Brinkman polytron® homogenizer (15-20 second bursts until all the material was in a uniform suspension) to produce a membrane protein preparation. Protein concentration was determined by the Bradford method (see WO 00/22131).

Candidate compounds preferably were screened using a 96-well plate format. Membrane protein preparations were diluted to 0.25 mg/ml in binding buffer to provide a final concentration of 12.5 μg/well in a 50 μl volume. One hundred μl of GDP buffer (37.5 ml of binding buffer and 2 mg GDP, Sigma Cat. No. G-7127) were added to each well followed by addition of a Wallac Scintistrip™ (Wallac). Five μl of a candidate compound were transferred into each well (i.e., 5 μl in a total assay volume of 200 μl resulting in a 1:40 ratio such that the final concentration of candidate was 10 pM). Fifty μl of membrane protein were added to each well (including a non-receptor containing membrane control) and pre-incubation was carried out for 5-10 minutes at room temperature. Thereafter, 50 μl of [³⁵S]GTPγS (0.6 nM) in binding buffer were added to each well, followed by incubation on a shaker for 60 minutes at room temperature. The assay was stopped by spinning the plates at 4,000 rpm for 15 minutes at 22° C. The plates then were aspirated with an 8 channel manifold, sealed with plate covers and read on a Wallac 1450™ using setting “Prot.#37” (as per manufacturer's instructions). Changes in the amount of material bound to the strips will determine whether the candidate was an inverse agonist (decrease relative to base line) or agonist (increase relative to base line).

What is claimed is:

1. A method of treating a condition associated with inflammation, the method comprising the step of administering to a subject an effective amount of compound of the following formula: 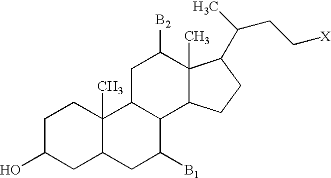
or a pharmaceutically acceptable salt, solvate, or hydrate thereof, wherein
B₁ and B₂ are each independently —OH, (C₁-C₆)alkyl, or —H;
X is Z-K;
Z is —CO or —CH₂;
K is —N(R₁)ₙ, —NR₁N(R₂)ₙ, —NR₁R₅R₂R₄, —N(R₁)ₚR₂NR₃R₄, or —N(R₁)ₚR₅R₂R₄ R₁, R₂, and R₃ are each independently (C₁-C₆)alkyl or absent;
n is from 0 to 3;
p is from 0 to 2;
R₄ is absent or is 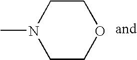
R₅ is absent or is

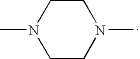

2. The method of claim 1, wherein the disease is selected from the group consisting of rheumatoid arthritis, bursitis, gout, polymyalgia rheumatica, allergic rhinitis, sinusitis, asthma, bronchiectasis, ulcerative colitis, Crohn's disease, silicosis, cachexia, cholecystitis, psoriasis, multiple sclerosis, systemic lupus erythematosus, thyroiditis, atherosclerosis, juvenile diabetes, graft versus host disease, meningitis, contact hypersensistivity, anaphylactic states, and chronic obstructive pulmonary disease.

3. The method of claim 1, wherein B₁ is —H, B₂ is —OH, Z is —CO and K is —NCH₂CH₂N⁺(CH₃)₃.

4. The method of claim 1, wherein B₁ is —H, B₂ is —OH, Z is —CO and K is

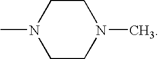

5. The method of claim 1, wherein B₁ is —H, B₂ is —OH, Z is —CO and K is

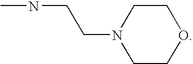

6. The method of claim 1, wherein the compound is administered in an effective amount of between about 0.01 mg and 10 mg per kg of body weight of the subject per day.

7. A method of treating a condition associated with inflammation, the method comprising the step of administering to a subject an effective amount of a prodrug of a compound of the following formula: 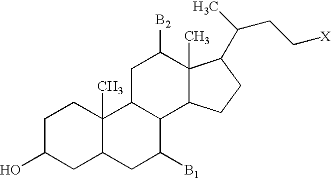
or a pharmaceutically acceptable salt, solvate, or hydrate thereof, wherein
B₁ and B₂ are each independently —OH, (C₁-C₆)alkyl, or —H;
X is Z-K;
Z is —CO or —CH₂;
K is —N(R₁)ₙ, —NR₁N(R₂)ₙ, —NR₁R₅R₂R₄, —N(R₁)ₚR₂NR₃R₄, or —N(R₁)ₚR₅R₂R₄ R₁, R₂, and R₃ are each independently (C₁-C₆)alkyl or absent;
n is from 0 to 3;
p is from 0 to 2;
R₄ is absent or is 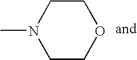
R₅ is absent or is

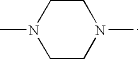

8. The method of claim 7, wherein the disease is selected from the group consisting of rheumatoid arthritis, bursitis, gout, polymyalgia rheumatica, allergic rhinitis, sinusitis, asthma, bronchiectasis, ulcerative colitis, Crohn's disease, silicosis, cachexia, cholecystitis, psoriasis, multiple sclerosis, systemic lupus erythematosus, thyroiditis, atherosclerosis, juvenile diabetes, graft versus host disease, meningitis, contact hypersensistivity, anaphylactic states, and chronic obstructive pulmonary disease.

9. The method of claim 7, wherein B₁ is —H, B₂ is —OH, Z is —CO and K is —NCH₂CH₂N⁺(CH₃)₃.

10. The method of claim 7, wherein B₁ is —H, B₂ is —OH, Z is —CO and K is

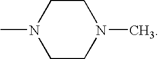

11. The method of claim 7, wherein B₁ is —H, B₂ is —OH, Z is —CO and K is

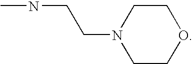

12. The method of claim 7, wherein the compound is administered in an effective amount of between about 0.01 mg and 10 mg per kg of body weight of the subject per day.

13. A method of activating GAVE10, the method comprising method comprising the step of administering to a subject an effective amount of compound of the following formula: 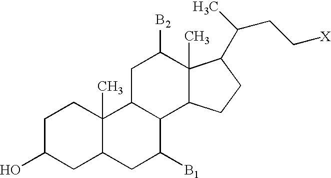
or a pharmaceutically acceptable salt, solvate, or hydrate thereof, wherein
B₁ and B₂ are each independently —OH, (C₁-C₆)alkyl, or —H;
X is Z-K;
Z is —CO or —CH₂;
K is —N(R₁)ₙ, —NR, N(R₂)ₙ, —NR₁R₅R₂R₄, —N(R₁)ₚR₂NR₃R₄, or —N(R₁)ₚR₅R₂R₄ R₁, R₂, and R₃ are each independently (C₁-C₆)alkyl or absent;
n is from 0 to 3;
p is from 0 to 2;
R₄ is absent or is 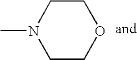
R₅ is absent or is

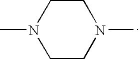

14. The method of claim 13, wherein B₁ is —H, B₂ is —OH, Z is —CO and K is —NCH₂CH₂N⁺(CH₃)₃.

15. The method of claim 13, wherein B, is —H, B₂ is —OH, Z is —CO and K is

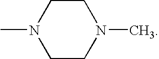

16. The method of claim 13, wherein B₁ is —H, B₂ is —OH, Z is —CO and K is

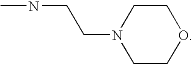

17. The method of claim 13, wherein the compound is administered in an effective amount of between about 0.01 mg and 10 mg per kg of body weight of the subject per day.

18. A method of activating GAVE10, the method comprising the step of administering to a subject an effective amount of a prodrug of a compound of the following formula: 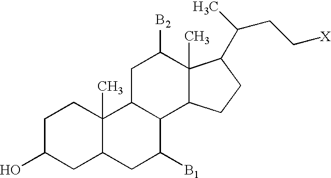
or a pharmaceutically acceptable salt, solvate, or hydrate thereof, wherein
B₁ and B₂ are each independently —OH, (C₁-C₆)alkyl, or —H;
X is Z-K;
Z is —CO or —CH₂;
K is —N(R₁)ₙ, —NR₁N(R₂)ₙ, —NR₁R₅R₂R₄, —N(R₁)ₚR₂NR₃R₄, or —N(R₁)ₚR₅R₂R₄ R₁, R₂, and R₃ are each independently (C₁-C₆)alkyl or absent;
n is from 0 to 3;
p is from 0 to 2;
R₄ is absent or is 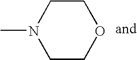
R₅ is absent or is

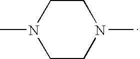

19. The method of claim 18, wherein B₁ is —H, B₂ is —OH, Z is —CO and K is —NCH₂CH₂N⁺(CH₃)₃.

20. The method of claim 18, wherein B₁ is —H, B₂ is —OH, Z is —CO and K is

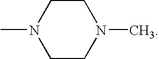

21. The method of claim 18, wherein B₁ is —H, B₂ is —OH, Z is —CO and K is

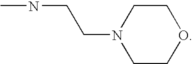

22. The method of claim 18, wherein the compound is administered in an effective amount of between about 0.01 mg and 10 mg per kg of body weight of the subject per day.

23. A method of inhibiting TNFα, the method comprising the step of administering to a subject an effective amount of a compound of the following formula: 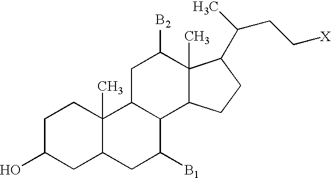
or a pharmaceutically acceptable salt, solvate, or hydrate thereof, wherein
B₁ and B₂ are each independently —OH, (C₁-C₆)alkyl, or —H;
X is Z-K;
Z is —CO or —CH₂;
K is —N(R₁)ₙ, —NR₁N(R₂)ₙ, —NR₁, R₅R₂R₄, —N(R₁)ₚR₂NR₃R₄, or —N(R₁)ₚR₅R₂R₄ R₁, R₂, and R₃ are each independently (C₁-C₆)alkyl or absent;
n is from 0 to 3;
p is from 0 to 2;
R₄ is absent or is 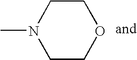
R₅ is absent or is

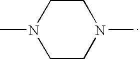

24. The method of claim 23, wherein B₁ is —H, B₂ is —OH, Z is —CO and K is —NCH₂CH₂N⁺(CH₃)₃.

24. The method of claim 23, wherein B₁ is —H, B₂ is —OH, Z is —CO and K is

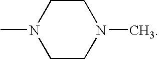

24. The method of claim 23, wherein B₁ is —H, B₂ is —OH, Z is —CO and K is

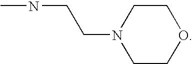

25. The method of claim 23, wherein the compound is administered in an effective amount of between about 0.01 mg and 10 mg per kg of body weight of the subject per day.

26. A method of inhibiting TNFα, the method comprising the step of administering to a subject an effective amount of a prodrug of a compound of the following formula: 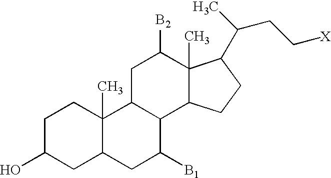
or a pharmaceutically acceptable salt, solvate, or hydrate thereof, wherein
B₁ and B₂ are each independently —OH, (C₁-C₆)alkyl, or —H;
X is Z-K;
Z is —CO or —CH₂;
K is —N(R₁)ₙ, —NR₁N(R₂)ₙ, —NR₁R₅R₂R₄, —N(R₁)ₚR₂NR₃R₄, or —N(R₁)PR₅R₂R₄ R₁, R₂, and R₃ are each independently (C₁-C₆)alkyl or absent;
n is from 0 to 3;
p is from 0 to 2;
R₄ is absent or is 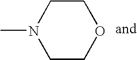
R₅ is absent or is

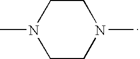

27. The method of claim 26, wherein B₁ is —H, B₂ is —OH, Z is —CO and K is —NCH₂CH₂N⁺(CH₃)₃.

28. The method of claim 26, wherein B₁ is —H, B₂ is —OH, Z is —CO and K is

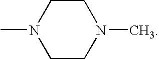

29. The method of claim 26, wherein B₁ is —H, B₂ is —OH, Z is —CO and K is

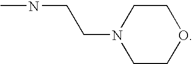

30. The method of claim 26, wherein the compound is administered in an effective amount of between about 0.01 mg and 10 mg per kg of body weight of the subject per day.

31. A method of inhibiting IL-6, the method comprising the step of administering to a subject an effective amount of a compound of the following formula: 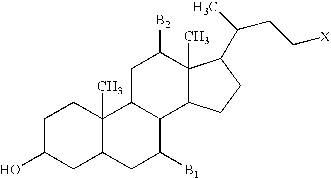
or a pharmaceutically acceptable salt, solvate, or hydrate thereof, wherein
B₁ and B₂ are each independently —OH, (C₁-C₆)alkyl, or —H;
X is Z-K;
Z is —CO or —CH₂;
K is —N(R₁)ₙ, —NR₁N(R₂)ₙ, —NR₁, R₅R₂R₄, —N(R₁)ₚR₂NR₃R₄, or —N(R₁)ₚR₅R₂R₄ R₁, R₂, and R₃ are each independently (C₁-C₆)alkyl or absent;
n is from 0 to 3;
p is from 0 to 2;
R₄ is absent or is 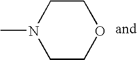
R₅ is absent or is

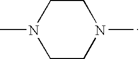

32. The method of claim 31, wherein B₁ is —H, B₂ is —OH, Z is —CO and K is —NCH₂CH₂N⁺(CH₃)₃.

33. The method of claim 31, wherein B₁ is —H, B₂ is —OH, Z is —CO and K is

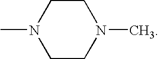

34. The method of claim 31, wherein B₁ is —H, B₂ is —OH, Z is —CO and K is

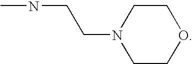

35. The method of claim 31, wherein the compound is administered in an effective amount of between about 0.01 mg and 10 mg per kg of body weight of the subject per day.

36. A method of inhibiting IL-6, the method comprising the step of administering to a subject an effective amount of a prodrug of a compound of the following formula: 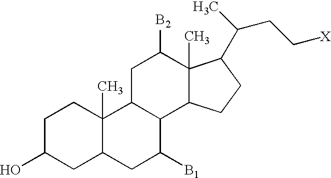
or a pharmaceutically acceptable salt, solvate, or hydrate thereof, wherein
B₁ and B₂ are each independently —OH, (C₁-C₆)alkyl, or —H;
X is Z-K;
Z is —CO or —CH₂;
K is —N(R₁)ₙ, —NR, N(R₂)ₙ, —NR₁, R₅R₂R₄, —N(R₁)ₚR₂NR₃R₄, or —N(R₁)ₚR₅R₂R₄ R₁, R₂, and R₃ are each independently (C₁-C₆)alkyl or absent;
n is from 0 to 3;
p is from 0 to 2;
R₄ is absent or is 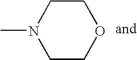
R₅ is absent or is

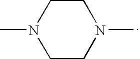

37. The method of claim 36, wherein B₁ is —H, B₂ is —OH, Z is —CO and K is —NCH₂CH₂N⁺(CH₃)₃.

38. The method of claim 36, wherein B₁ is —H, B₂ is —OH, Z is —CO and K is

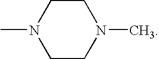

39. The method of claim 36, wherein B₁ is —H, B₂ is —OH, Z is —CO and K is

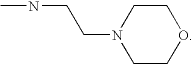

40. The method of claim 36, wherein the compound is administered in an effective amount of between about 0.01 mg and 10 mg per kg of body weight of the subject per day.
